(19) United States
(12) Patent Application Publication
(10) Pub. No.: US 2018/0042762 A1
(43) Pub. Date: Feb. 15, 2018

(54) THERMAL SYSTEM

(71) Applicant: Stryker Corporation, Kalamazoo, MI (US)

(72) Inventor: James K. Galer, Byron Center, MI (US)

(21) Appl. No.: 15/675,066

(22) Filed: Aug. 11, 2017

Related U.S. Application Data
(60) Provisional application No. 62/373,564, filed on Aug. 11, 2016.

(51) Int. Cl. A61F 7/00; A47C 21/04; A61F 7/08

(52) U.S. Cl. CPC A61F 7/0085; A61F 7/08; A47C 21/042; A61F 7/007; A61F 2007/0001; G01K 13/002

(57) ABSTRACT

A thermal system includes a thermal control unit adapted to supply temperature controlled fluid to one or more thermal pads. The thermal pads may include an interior chamber for fluid circulation, an inlet and an outlet, and first and second temperature sensors positioned adjacent the inlet and outlet, respectively. Some pads include a valve adapted to control an amount of fluid flowing through one or more channels within the pad. The thermal control unit may include a controller that controls the fluid temperature based upon temperature readings from first and second temperature sensors positioned remotely from the thermal control unit. The thermal control unit may also include a valve that selectively controls the amount of fluid exiting the thermal control from one or more outlets based upon one or more temperature readings. The system may include one or more thermocouples with conductors placed on or in the thermal pads.

### CROSS-REFERENCE TO RELATED APPLICATIONS

This application claims priority to U.S. provisional patent application Ser. No. 62/373,564 filed Aug. 11, 2016, by inventor and entitled THERMAL SYSTEM, the complete disclosure of which is incorporated herein by reference.

### BACKGROUND

The present disclosure relates to a thermal control system for controlling the temperature of circulating fluid that is delivered to one or more thermal pads positioned in contact with a patient.

Thermal control systems are known in the art for controlling the temperature of a patient by providing a thermal control unit that supplies temperature controlled fluid to one or more thermal pads positioned in contact with a patient. The thermal control unit includes one or more heat exchangers for controlling the temperature of the fluid and a pump that pumps the temperature controlled fluid to the pad(s). After passing through the pad(s), the fluid is returned to the thermal control unit where any necessary adjustments to the temperature of the returning fluid are made before being pumped back to the pad(s). In some instances, the temperature of the fluid is controlled to a target temperature, while in other instances the temperature of the fluid is varied as necessary in order to automatically effectuate a target patient temperature. The thermal control unit can therefore be used to warm or cool a patient.

The pads are placed in close contact with the patient in order to facilitate heat exchange between the patient and the pad. In one common arrangement, three pads are applied to the patient: one applied around the patient's torso, one applied around the patient's right leg, and one applied around the patient's left leg.

### SUMMARY

The present disclosure provides various improved aspects to a thermal control system, including the thermal control unit and the thermal pads. These improved aspects include improved thermal transfer between the pads and the patient; improved control of the patient's temperature; improved sensing of the temperature environment at the patient; and improved targeting of the temperature controlled fluid to one or more areas of the patient. Other improved aspects of the present disclosure are described in more detail below.

According to one embodiment of the present disclosure, a thermal pad for controlling a patient's temperature is provided that includes a body, a first temperature sensor, a second temperature sensor, and a temperature output. The body is adapted to be placed in contact with the patient and defines an interior in which fluid circulates. The body includes a fluid inlet adapted to receive fluid from a thermal control unit adapted to control a temperature of the fluid, and a fluid outlet adapted to return the fluid to the thermal control unit. The first temperature sensor is positioned adjacent the fluid outlet and adapted to detect a temperature of the fluid exiting the thermal pad. The second temperature sensor is positioned adjacent the fluid inlet and adapted to detect a temperature of the fluid entering the thermal pad. The temperature output reports the temperature of the first and second temperature sensors to the thermal control unit.

According to other aspects of the disclosure, the thermal pad includes a second fluid inlet and a second fluid outlet that are in communication with a fluid channel inside of the thermal pad that is fluidly separated from a fluid channel that receives fluid from the first inlet. The fluid in the channels therefore does not mix while inside the thermal pad. Third and fourth temperature sensors may also be included that detect temperature of the fluid entering and exiting the thermal pad via the second fluid inlet and outlet, respectively.

In some embodiments, the thermal pad includes an interior surface adapted to contact the patient and an exterior surface adapted to face away from the patient wherein the interior surface includes a plurality of craters. The craters create suction when the interior surface is applied to a skin of the patient and the suction releasably retains the interior surface against the patient's skin. The suction allows the thermal pad to be retained against the skin of the patient without using an adhesive.

Alternatively, the interior surface may include a gel layer adapted to directly contact a skin of the patient and to have inherent adhesive properties for releasably adhering to the patient's skin.

According to another embodiment, a thermal pad for controlling a patient's temperature is provided. The thermal pad includes a body, at least one channel, and a valve. The body is adapted to be placed in contact with the patient and includes an interior in which fluid circulates. The body further includes an inlet adapted to receive temperature controlled fluid from a thermal control unit and an outlet adapted to return the fluid to the thermal control unit. The channel is in the body and in fluid communication with the inlet and outlet. The valve is adapted to control an amount of fluid flowing through the channel.

According to other aspects of the disclosure, a port is included that is adapted to receive a control signal for controlling the valve.

The pad may further include a plurality of channels wherein a first one of the channels is associated with a first zone of the thermal pad and a second one of the channels is associated with a second zone of the thermal pad. In such cases, the valve controls what proportion of the fluid from the fluid inlet is directed to the first zone versus the second zone.

In some embodiments, the valve is positioned at the fluid inlet and controls the amount of fluid flowing through the channel.

The valve is a pressure operated valve controlled by a pressure of the fluid in some embodiments. In other embodiments, the valve is controlled by signals that are based upon, either wholly or partially, a fluid flow rate and/or volume.

According to another embodiment, a thermal control unit is provided for supplying fluid controlled temperature to one or more thermal pads. The thermal control unit includes a fluid outlet, a fluid inlet, a heat exchanger, a pump, and a controller. The fluid outlet couples to a fluid supply line and the fluid inlet couples to a fluid return line. The pump circulates fluid from the fluid inlet through the heat exchanger and to the fluid outlet. The controller receives a first temperature reading from a first temperature sensor positioned remote from the thermal control unit and adapted to measure a temperature of the fluid delivered by the fluid supply line to a thermal pad. The controller also receives a second temperature reading from a second temperature sensor positioned remote from the thermal control unit and adapted to measure a temperature of the fluid returned from the thermal pad by the fluid return line. The controller controls the heat exchanger based upon the first and second temperature readings.

According to other aspects, the controller receives third and fourth temperature readings from third and fourth temperature sensors that are positioned remotely from the thermal control unit. The third temperature sensor measures a temperature of the fluid delivered by a second fluid supply line to the thermal pad and the fourth temperature sensor measures a temperature of the fluid returned from the thermal pad to a second fluid return line. The controller controls the heat exchanger based upon the third and fourth temperature readings.

The thermal control unit includes, in some embodiments, one or more valves that control the amount of fluid flowing to the one or more fluid supply lines. The controller is adapted to control the valves based upon temperature readings from the multiple temperature sensors.

In some embodiments, the controller determines a first difference between the first and second temperature readings and a second difference between the third and fourth temperature readings. The controller controls at least one valve based upon the first and second differences. The controller may also or alternatively control a speed of the pump based upon one or more of the temperature differences.

According to another embodiment, a thermal control unit is provided that includes first and second fluid inlets, first and second fluid outlets, a heat exchanger, a pump, a temperature sensor, a valve, and a controller. The pump circulates fluid from the first and second fluid inlets through the heat exchanger and to the first and second fluid outlets. The temperature sensor detects a temperature of fluid returning from one of the first or second fluid inlets. The valve controls an amount of fluid flowing to the first fluid outlet. The controller controls the valve based at least in part upon the temperature sensed by the temperature sensor.

In some embodiments, the thermal control unit further includes a second valve for controlling an amount of fluid flowing to the second fluid outlet. The controller controls the second valve based at least in part upon the temperature sensed by one or more of the temperature sensors.

The controller may also, or alternatively, control one or more of the valves based upon one or more temperature readings from temperature sensors positioned remote from the thermal control unit.

The first fluid supply line and first fluid return line are coupled to a first zone of a thermal pad, in some embodiments, and the second fluid supply line and the second fluid return line are coupled to a second zone of the thermal pad.

According to another embodiment, a thermal control system is provided that includes a thermal control unit, a thermal pad, first, second, third, and fourth hoses, and a thermocouple. The thermal control unit includes first and second fluid outlets and first and second fluid inlets, a heat exchanger, a pump, and a controller. The thermal pad is adapted to receive fluid from the thermal control unit. The first and second hoses couple the first and second fluid outlets, respectively, to a first zone of the thermal pad. The third and fourth hoses couple the first and second fluid inlets, respectively, to a second zone of the thermal pad. The thermocouple has a first and second conductor coupled to a first location of the first zone. The thermocouple communicates with the controller of the thermal control unit.

According to other aspects, the controller controls the heat exchanger based upon at least an output of the thermocouple. The controller may also or alternatively control a speed of the pump and/or a valve based upon outputs from the thermocouple.

Before the various embodiments disclosed herein are explained in detail, it is to be understood that the claims are not to be limited to the details of operation or to the details of construction, nor to the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a thermal control system according to one aspect of the present disclosure shown applied to a patient on a patient support apparatus;

FIG. 2 is block diagram of the thermal control system of FIG. 1;

FIG. 3 is a plan view of a first embodiment of a thermal pad usable with the thermal control system of FIG. 1;

FIG. 4 is an exploded perspective view of the thermal pad of FIG. 3;

FIG. 5 is a diagram of an illustrative flow channel layout that may be incorporated into the inside of the thermal pad of FIG. 3;

FIG. 6 is a plan view of a second embodiment of a thermal pad usable with the thermal control system of FIG. 1;

FIG. 7 is a plan view of a third embodiment of a thermal pad usable with the thermal control system of FIG. 1;

FIG. 8 is a diagram of an illustrative flow channel layout that may be incorporated into the inside of the thermal pads of FIG. 3 or 7;

FIG. 9 is a plan view of a fourth embodiment of a thermal pad usable with the thermal control system of FIG. 1;

FIG. 10 is an exploded perspective view of the thermal pad of FIG. 9;

FIG. 11 is a side sectional view of the thermal pad of FIG. 9; and

FIG. 12 is a side view of an illustrative thermal pad interior layer that may be incorporated into any of the thermal pads disclosed herein.

### DETAILED DESCRIPTION OF THE EMBODIMENTS

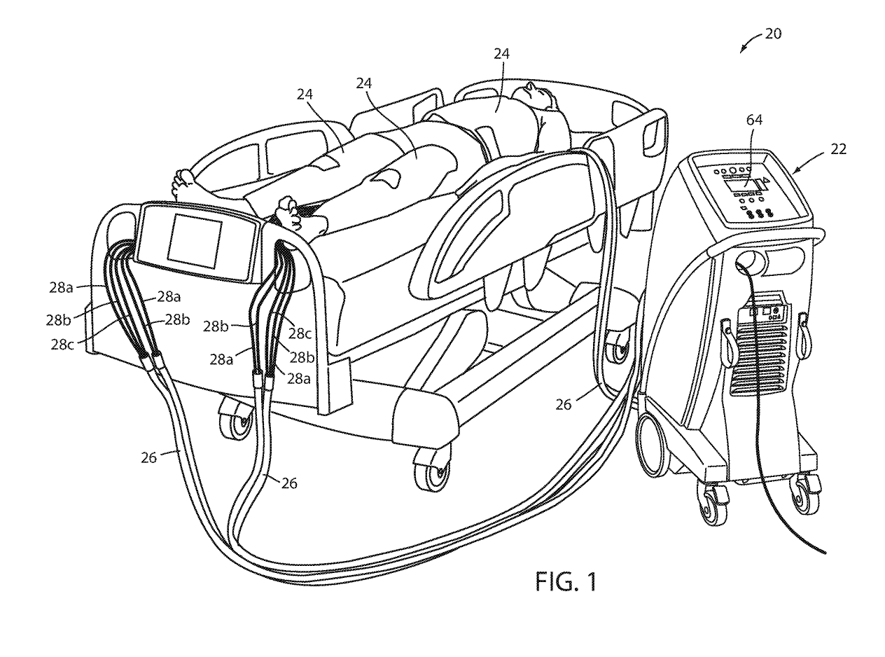

A thermal control system 20 according to one embodiment of the present disclosure is shown in FIG. 1. Thermal control system 20 is adapted to control the temperature of a patient 30, which may involve raising, lowering, or maintaining the patient's temperature, or combinations thereof. Thermal control system 20 includes a thermal control unit 22 coupled to one or more thermal therapy devices 24. The thermal therapy devices 24 are illustrated in FIG. 1 to be thermal pads, but it will be understood that thermal therapy devices 24 may take on other forms, such as, but not limited to, blankets, vests, patches, caps, or other structure. For purposes of the following written description, thermal therapy devices 24 will be referred to as thermal pads 24, but it will be understood by those skilled in the art that this terminology is used merely for convenience and that the phrase “thermal pad” is intended to cover all of the different variations of thermal therapy devices 24 mentioned above (e.g. blankets, vests, patches, caps, etc.).

Thermal control unit 22 is coupled to thermal pads 24 via a plurality of hoses 26. Each hose includes one or more lines 28. In the embodiment shown in FIG. 1, each hose 26 includes a fluid supply line 28a, a fluid return line 28b, and one or more control lines 28c. Thermal control unit 22 delivers temperature controlled fluid (such as, but not limited to, water) to the thermal pads 24 via the fluid supply lines 28a. After the temperature controlled fluid has passed through thermal pads 24, thermal control unit 22 receives the temperature controlled fluid back from thermal pads 24 via the return lines 28b. Control lines 28c are used by thermal control unit 22 in different manners, depending upon the capabilities of thermal control unit 22, the construction of one or more of the thermal pads 24, and/or the desired treatment to be applied to the patient 30. As will be discussed in greater detail below, in some instances thermal control unit 22 uses a control line 28c to take one or more temperature readings from a location adjacent to, or inside of, thermal pad 24. In other instances, thermal control unit 22 uses a control line 28c to control one or more valves incorporated into thermal pad 24. In still other instances, thermal control unit 22 uses control lines 28c to both take temperature readings and control one or more valves inside of thermal pads 24. Still other uses are discussed below.

In the embodiment of thermal control system 20 shown in FIG. 1, three thermal pads 24 are used in the treatment of patient 30. A first thermal pad 24 is wrapped around a patient's torso, while second and third thermal pads 24 are wrapped, respectively, around the patient's right and left legs. Other configurations can be used and different numbers of thermal pads 24 may be used with thermal control unit 22, depending upon the number of inlet and outlet ports that are included with thermal control unit 22. By controlling the temperature of the fluid delivered to thermal pads 24 via supply lines 28a, the temperature of the patient 30 can be controlled via the close contact of the pads 24 with the patient 30 and the resultant heat transfer therebetween.

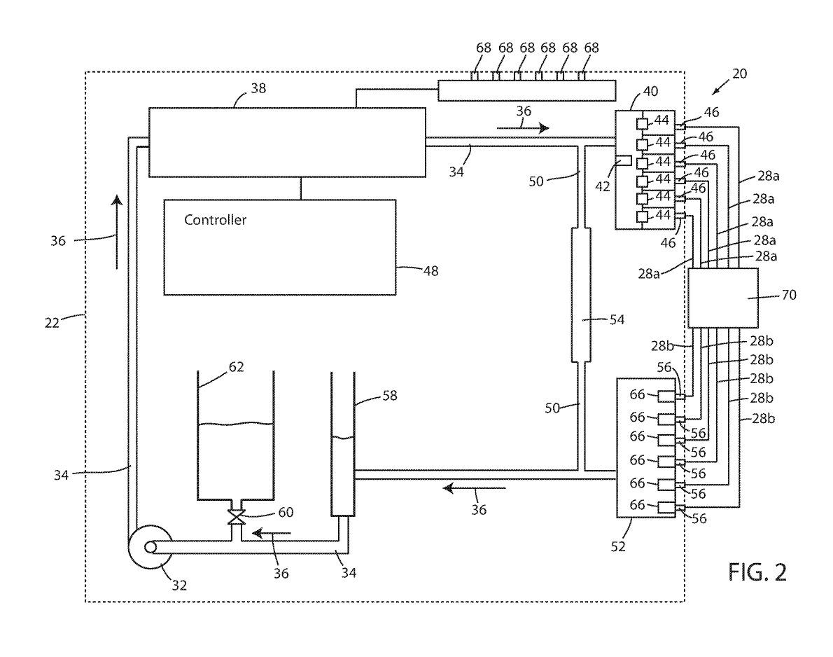

Thermal control unit 22 is adapted to raise or lower the temperature of the fluid supplied to thermal pads 24 via supply lines 28a. As shown in FIG. 2, thermal control unit 22 includes a pump 32 for circulating fluid through a circulation channel 34. Pump 32, when activated, circulates the fluid through circulation channel 34 in the direction of arrows 36 (clockwise in FIG. 2). Starting at pump 32 the circulating fluid first passes through a heat exchanger 38 where it is delivered to an outlet manifold 40 having an outlet temperature sensor 42, a plurality of valves 44, and a plurality of outlet ports 46. Temperature sensor 42 is adapted to detect a temperature of the fluid inside of outlet manifold 40 and report it to a controller 48. Valves 44 are adapted to move between open and closed positions (and in some embodiments, one or more positions therebetween) under the control of controller 48. Outlet ports 46 are adapted to be coupled to supply lines 28a of thermal control system 20 and deliver temperature controlled fluid thereto. Valves 44 control how much fluid flows from outlet manifold 40 to each of the supply lines 28a, as will be discussed in greater detail below. Supply lines 28a are, in turn, coupled to a thermal load 70. Thermal load 70 includes one or more thermal pads 24 that are used to control the temperature of a patient 30.

Control unit 22 also includes a bypass line 50 fluidly coupled to outlet manifold 40 and an inlet manifold 52. Bypass line 50 allows fluid to circulate through circulation channel 34 even in the absence of any thermal pads 24 or lines 28a being coupled to any of outlet ports 46. In the illustrated embodiment, bypass line 50 includes an optional filter 54 that is adapted to filter the circulating fluid. If included, filter 54 may be a particle filter adapted to filter out particles within the circulating fluid that exceed a size threshold, or filter 54 may be a biological filter adapted to purify or sanitize the circulating fluid, or it may be a combination of both.

Inlet manifold 52 includes the plurality of inlet ports 56 that receive fluid returning from the one or more connected thermal pads 24. The incoming fluid from inlet ports 56, as well as the fluid passing through bypass line 50, travels back toward the pump 32 into an air separator 58. Air separator 58 includes any structure in which the flow of fluid slows down sufficiently to allow air bubbles contained within the circulating fluid to float upwardly and escape to the ambient surrounding. In some embodiments, air separator 58 is constructed in accordance with any of the configurations disclosed in commonly assigned U.S. patent application Ser. No. 62/361,124 filed Jul. 12, 2016, by inventor Gregory S. Taylor and entitled THERMAL CONTROL SYSTEM, the complete disclosure of which is hereby incorporated herein by reference. After passing through air separator 58, the circulating fluid flows past a valve 60 positioned beneath a fluid reservoir 62 that supplies fluid to thermal control unit 22. After passing by valve 60, the circulating fluid travels to pump 32 and the circuit is repeated.

Controller 48 of thermal control unit 22 is contained within a main body of thermal control unit 22 and is in electrical communication with a variety of different sensors and/or actuators. More specifically, controller 48 is in electrical communication with pump 32, heat exchanger 38, a control panel 64 (FIG. 1), outlet temperature sensor 42, valves 44, a plurality of inlet temperature sensors 66, and a plurality of control ports 68.

Inlet temperature sensors 66 are positioned inside of inlet manifold 52 and measure the temperature of the fluid returning through each of inlet ports 56. That is, each temperature sensor 66 measures the temperature of fluid entering one of the inlet ports 56. One or more of the inlet ports 56, in turn, are coupled to return lines 28b that return fluid from thermal load 70 to thermal control unit 22.

Control panel 64 allows a user to operate thermal control unit 22, including setting a desired fluid target temperature and/or a desired patient target temperature, and/or to control other aspects of thermal control unit 22. Control panel 64 communicates with controller 48 and includes controls enabling a user to turn control unit 22 on and off, as well as one or more controls enabling the user to select a target temperature for the fluid delivered to thermal pads 24. In some embodiments, control panel 64 also allows a user to select a target temperature for the patient being treated, rather than a specific target temperature for the fluid. When this feature is present, controller 48 makes automatic adjustments to the temperature of the fluid in order to bring the patient's temperature to the desired patient target temperature. In addition, control panel 64 allows a user to configure how thermal control unit 22 controls the thermal pads in light of temperature and/or flow information received by thermal control unit 22, as will be discussed more below. Control panel 64 also allows a user to configure how thermal control unit 22 controls one or more valves that may be positioned inside of one or more thermal pads and/or inside of thermal control unit 22.

Both the outlet temperature sensor 42 and one or more of the inlet temperature sensors 66 may be used to provide feedback to controller 48, depending upon the embodiment of thermal control unit 22, so that controller 48 can adjust heat exchanger 38, as appropriate, in order to effectuate closed-loop control of the temperature of the circulating fluid.

Each control port 68 couples to a control line 28c. In other embodiments discussed below, control ports 68 received temperature readings via control lines 28c from one or more locations within one or more thermal pads 24, as will be discussed below. In other embodiments, control ports 68 output one or more control signals to one or more valves positioned inside of one or more thermal pads 24. In still other embodiments, control ports 68 are used for both receiving temperature readings and for controlling valves.

Controller 48 includes any and all electrical circuitry and components necessary to carry out the functions and algorithms described herein, as would be known to one of ordinary skill in the art. Generally speaking, controller 48 may include one or more microcontrollers, microprocessors, and/or other programmable electronics that are programmed to carry out the functions described herein. It will be understood that controller 48 may also include other electronic components that are programmed to carry out the functions described herein, or that support the microcontrollers, microprocessors, and/or other electronics. The other electronic components include, but are not limited to, one or more field programmable gate arrays, systems on a chip, volatile or nonvolatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions in thermal control unit 22, or they may reside in a common location within thermal control unit 22. When physically distributed, the components may communicate using any suitable serial or parallel communication protocol, such as, but not limited to, CAN, LIN, Firewire, I-squared-C, RS-232, RS-485, universal serial bus (USB), etc.

Controller 48 uses the outputs of outlet temperature sensor 42 and the outputs from one or more of the inlet temperature sensors 66 and/or one or more thermal pad temperature sensors (which are communicated via control lines 28c and control ports 68) to control heat exchanger 38 and/or pump 32. Controller 48 controls the heat exchanger and/or pump 32 such that the temperature of the circulating fluid has its temperature adjusted (or maintained) in accordance with the operating mode (manual or automatic) selected by the user of thermal control unit 22. Controller 48 may control the temperature of the fluid using a closed-loop proportional-integral (PI) controller, a closed-loop proportional-integral-derivative (PID), controller, or some other type of closed-loop controller.

Control unit 22 may also be modified to include one or more flow sensors that measure the rate of fluid flow and report this information to controller 48. In such modified embodiments, controller 48 uses the flow rate in determining what control signals to send to heat exchanger 38, valves 44, pump 32, and/or one or more thermal pads via control ports 68.

It will be understood by those skilled in the art that the particular order of the components along circulation channel 34 of control unit 22 may be varied from what is shown in FIG. 2. For example, although FIG. 2 depicts pump 32 as being upstream of heat exchanger 38 and air separator 58 as being downstream of heat exchanger 38, this order may be changed. Air separator 58, pump 32, heat exchanger 38 and reservoir 62 may be positioned at any suitable location along circulation channel 34. Indeed, in some embodiments, reservoir 62 is moved so as to be in line with and part of circulation channel 34, rather than external to circulation channel 34 as shown in FIG. 2.

Further details regarding the construction and operation of one embodiment of thermal control unit 22 that are not described herein are found in commonly assigned U.S. patent application Ser. No. 14/282,383 filed May 20, 2014, inventors Christopher Hopper et al. and entitled THERMAL CONTROL SYSTEM, the complete disclosure of which is incorporated herein by reference.

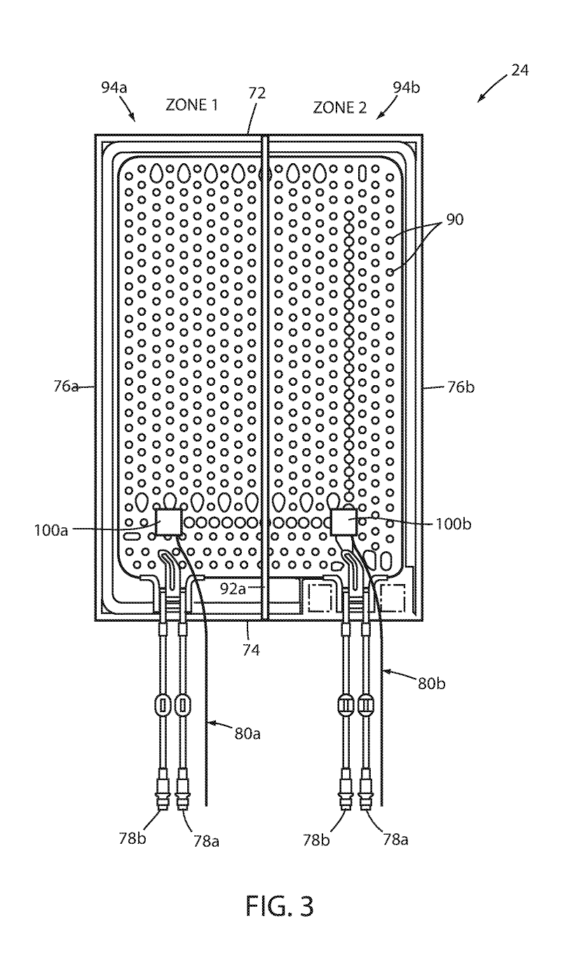

FIG. 3 illustrates in greater detail one embodiment of a thermal pad 24 that may be used with the thermal control system 20 of FIG. 1. Thermal pad 24 includes a top 72, a bottom 74, a first side 76a and a second side 76b that collectively define a perimeter of thermal pad 24. Thermal pad 24 also includes a fluid inlet hose 78a, a fluid outlet hose 78b, and a control line 80. Fluid inlet hose 78a is adapted to couple to fluid supply line 28a from thermal control unit 22. Fluid outlet hose 78b is adapted to couple to fluid return line 28b from thermal control unit 22. Control line 80 is adapted to couple to control line 28c from thermal control unit 22.

Although not shown, thermal pad 24 may also include one or more straps that are used to secure thermal pad 24 to patient 30 when in use. Each strap may be adapted to releasably attach to another strap after thermal pad 24 has been wrapped around the patient 30. In some embodiments, the straps include hook and loop type fasteners, such as those sold under the tradename Velcro, while in other embodiments, the straps include one or more repositionable tapes. In other embodiments, the straps include other types of fasteners for securing themselves to each other in order to maintain pad 24 in a wrapped stated around the patient's leg or torso.

Although thermal pad 24 of FIG. 3 is shown as having a generally rectangular shape, it will be understood by those skilled in the art that this may be varied greatly. That is, thermal pad 24 may take on any shape that is conducive to being wrapped around one or more portions of patient 30. In some embodiments, those thermal pads 24 that are intended to be wrapped around the patient's torso have a different shape than those intended to be wrapped around the patient's legs. Those adapted to be wrapped around the patient's legs may include one or more cutouts or contours that allow the patient to bend his or her knees while thermal pads 24 are wrapped around his or her legs.

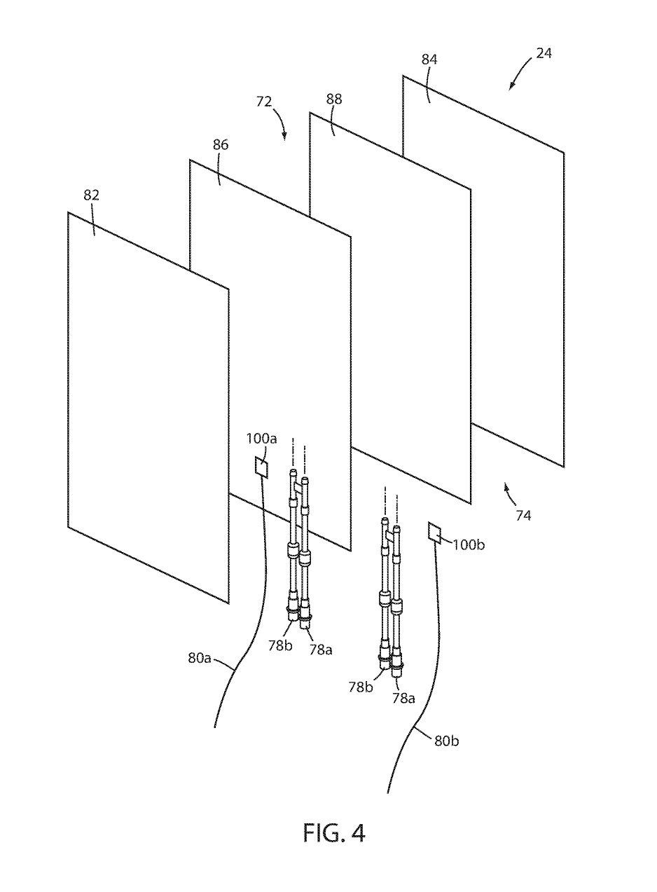

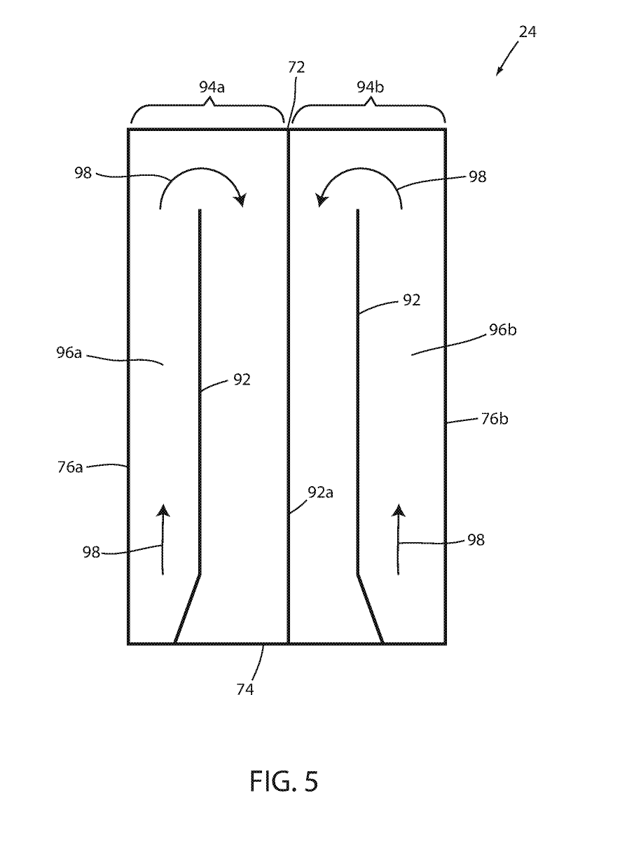

FIGS. 4 and 5 illustrate in greater detail the internal construction of thermal pad 24 of FIG. 3. As shown more clearly in FIG. 4, thermal pad 24 includes an interior layer 82, an exterior layer 84, a first intermediate layer 86, and a second intermediate layer 88. In some embodiments, one or both of interior layer 82 and exterior layer 84 are omitted. Interior layer 82 is designed to face toward the patient 30 while exterior layer 84 is designed to face away from the patient 30. Interior layer 82, first and second intermediate layers 86 and 88, and exterior layer 84 are all bonded to each other around their perimeters. Exterior layer 84 is an insulating layer that is bonded to second intermediate layer 88 over substantially the entire exterior surface of exterior layer 84. Interior layer 82 and intermediate layers 86 and 88 may all be constructed from any suitable plastic material that is flexible enough to conform to the patient's body and that provides good thermal conductivity. In some embodiments, interior layer 82 and intermediate layers 86 and 88 are constructed from a polyester and/or nylon composite. Other materials, however, may be used. Exterior layer 84 is constructed from any suitably flexible material that has relatively poor thermal conductivity properties so as to thermally insulate the other layers (and the fluid contained therein) from the temperature of the ambient surroundings. In some embodiments, exterior layer 84 is constructed from material that includes a polyester foam, or other type of foam. Still other constructions are possible.

The bonding of interior layer 82, exterior layer 84, and intermediate layers 86 and 88 to each other about their periphery may be accomplished in any suitable manner. In some embodiments, the bonding is carried out using welds. Such welds may be implemented via heat welding, ultrasonic welding, Radio Frequency (RF) welding, or by other types of welding. In addition to being bonded to each other around their perimeters, first intermediate layer 86 and second intermediate layer 88 are bonded to each other at a plurality of internal locations 90 (FIG. 3). The space between first intermediate layer 86 and second intermediate layer 88 where they are not bonded to each other defines a fluid chamber in which the temperature controlled fluid supplied by thermal control unit 22 (via supply line 28a) circulates.

In some regions of thermal pad 24, bonding locations 90 are contiguous with each other to create one or more fluid channel walls 92 within thermal pad 24. One such wall 92a extends contiguously from top 72 to bottom 74 to thereby separate thermal pad 24 into a first zone 94a and a second zone 94b. First zone 94a is fluidly isolated from second zone 94b. That is, fluid that is supplied to first zone 94a by the left inlet hose 78a in FIG. 3 does not mix inside of pad 24 with the fluid that is supplied to second zone 94b by the right inlet hose 78a in FIG. 78b. Instead, all of the temperature controlled fluid that is supplied to thermal pad 24 by the left inlet hose 78a (FIG. 3) circulates through first zone 94a and eventually exits pad 24 via the left hose 78b (FIG. 3). Likewise, all of the temperature controlled fluid that is supplied to thermal pad 24 by the right inlet hose 78a (FIG. 3) circulates through second zone 94b and eventually exits pad 24 via the right hose 78b (FIG. 3).

Although FIG. 3 illustrates first and second zone 94a and 94b as both being rectangular and substantially the same size, it will be understood that this is merely one manner of implementing zones 94a and 94b. In other embodiments, zones 94a and 94b are not symmetrical and/or are not of equal size. In some embodiments, one or more of zones 94a and 94b have shapes, sizes, and/or locations that are defined with reference to their intended anatomical placement on the patient. For example, first zone 94a may be sized, shaped, and positioned on thermal pad 24 such that it will contact a first particular anatomical location of the patient and second zone 94b may be sized, shaped, and/or positioned on thermal pad 24 such that it will contact a second anatomical location of the patient when thermal pad 24 is placed in contact with the patient.

The paths followed by the temperature controlled fluid while it circulates inside of thermal pad 24 may vary. FIG. 5 illustrates one illustrative layout of a first fluid channel 96a and a second fluid channel 96b. Fluid flows in channels 96a and 96b in the directions indicated by arrows 98. First fluid channel 96a is defined in first zone 94a and second fluid channel 96b is defined in second zone 94b. First and second fluid channels 96a and 96b are each defined essentially as an inverted “U” in FIG. 5. It will be understood by those skilled in the art that different shapes of fluid channels 96a and 96b can be used. Further, it will be understood by those skilled in the art that, in some embodiments, fluid channel 96a has a shape and/or layout that is different from the shape and/or layout of fluid channel 96b. Still further, in some embodiments, first zone 94a and/or second zone 94b can be comprised of multiple fluid channels 96 that may be arranged in series or parallel, or any combination or sequence of channels arranged in parallel or serial fashion.

As shown in FIG. 3, thermal pad 24 further includes two control lines 80a and 80b. In this embodiment, a first one of the control lines 80a is coupled to a first temperature sensor 100a and a second one of the control lines 80b is coupled to a second temperature sensor 100b. Temperature sensors 100a and 100b are thermocouples, in at least one embodiment. In other embodiments, temperature sensors 100a and/or 100b are thermistors. In still other embodiments, temperature sensors 100a and/or 100b are implemented as still other types of temperature sensors.

First temperature sensor 100a is integrated into thermal pad 24 at a first location that, in at least one embodiment, is positioned close to fluid outlet hose 78b coupled to first zone 94a. First temperature sensor 100a therefore provides a temperature reading of the temperature controlled fluid after it has circulated through substantially all of the first zone 94a of thermal pad 24. Second temperature sensor 100b is integrated into thermal pad 24 at a second location that, in at least one embodiment, is positioned close to fluid outlet hose 78b coupled to second zone 94b. Second temperature sensor 100b therefore provides a temperature reading of the temperature controlled fluid after it has circulated through substantially all of the second zone 94b of thermal pad 24.

In other embodiments, temperature sensors 100a and/or 100b may be positioned at other locations inside of thermal pad 24. For example, temperature sensors 100a and/or 100b may be positioned at any location along fluid channels 96a and 96b, respectively. Regardless of the position of temperature sensors 100a and 100b, they are secured inside of thermal pad 24 such that their location does not change. Any conventional manner of securing them inside of thermal pads 24 may be used, such as, but not limited to, the use of adhesive, fasteners, welding, etc.

When temperature sensors 100a and 100b are positioned close to fluid outlet hoses 78b of their respective zones 94a and 94b, temperature sensors 100a and 100b report temperature readings back to thermal control unit 22 (via control ports 68) that are indicative of how much thermal energy has been transferred to or from the patient. In some embodiments, thermal control unit 22 is constructed to include a flow meter coupled to each of inlet ports 56 or each of outlet ports 46 so that controller 48 can calculate how much fluid is circulating through each zone 94 and/or pad 24. By knowing the amount of fluid returning from a zone 94 and/or pad 24, the temperature of the returning fluid (measured by temperatures sensors 100a and 100b), and the temperature delivered to each pad or zone 94 (measured by outlet temperature sensor 42), controller 48 is able to calculate how much thermal energy has been delivered or absorbed by each zone 94 and/or pad 24. In some embodiments of thermal control unit 22, controller 48 calculates this thermal energy and displays it to the user. In still other embodiments, one or more flow meters may be incorporated into the thermal pad 24 and/or one or more of the hoses 26 that couple thermal pad 24 to thermal control unit 22.

In other embodiments of thermal control unit 22, controller 48 calculates this thermal energy, compares the calculated thermal energies of each zone 94 and/or pad 24 to each other, and determines if any discrepancies exist that exceed one or more thresholds. Such discrepancies may be indicative of poor contact of a zone or pad with the patient, or poor circulation of fluid through one or more zones or pads 24. For example, if controller 48 determines that a first thermal pad 24 coupled to one of the patient's legs is absorbing X amount of kilocalories per hour and a second thermal pad 24 coupled to the patient's other leg is absorbing Y amount of kilocalories per hour, and X is significantly different from Y, there is likely an issue with one of the thermal pads 24. This is because there typically is not a large difference between the patient's right and left legs in terms of the amount of heat they can deliver or absorb. As noted, the issue may be due to one of the thermal pads 24 not being wrapped properly around the patient's leg, or it may be due to reduced fluid flow, such as from a constricted fluid line, or it may be due to other reasons. By notifying the user of the potential issue, thermal control unit 22 is able to prompt the user to take investigative action in order to address and correct the issue.

In some embodiments, thermal control unit 22 does not include flow meters for each individual inlet port 56 and therefore does not calculate how much thermal energy is being delivered or absorbed by a thermal pad 24 and/or its individual zones 94. In such embodiments, controller 48 still monitors differences in temperatures sensed by temperature sensors 100a and 100b and issues an alert if those temperature differences exceed one or more predefined thresholds. Thus, for example, if fluid if being delivered to both zones 94a and 94b that is at approximately the same temperature, yet temperature sensor 100a is sensing a temperature that differs from the temperature sensed by temperature sensor 100b that is greater than a threshold, controller 48 issues an alert to the user to investigate the possible cause of this greater-than-normal temperature difference.

In still other embodiments, thermal control unit 22 is configured to monitor the flow rates associated with different thermal pads 24 and/or different zones of one or more thermal pads 24 and determine if any of the monitored flow rates differ from each other by more than one or more thresholds. This monitoring is carried out independently of the temperature monitoring. Thus, for example, if thermal control unit 22 detects that the temperature of the returning fluid from first and second pads 24 is the same (or has very little difference), yet one of the pads has a markedly higher or lower flow rate, this may be an indication that one or both of the thermal pads 24 are not properly positioned on the patient. Consequently, if thermal control unit 22 detects differences in flow rates that exceed one or more predetermined thresholds, it provides notification to the user to investigate the source of such differences.

By positioning temperature sensors 100a and 100b inside of thermal pad 24, more accurate indications of the temperature of the fluid at the local region where the heat transfer is occurring between pad 24 and the patient 30 are able to be obtained. That is, measurements of the temperature of the fluid inside thermal control unit 22, either after it has returned from the thermal pad or when it is inside outlet manifold 40 and ready to be delivered to the pads, are not necessarily accurate indications of the temperature of the fluid at the thermal pad 24. Differences in these temperatures can exist due to thermal loss or gain that occurs as the fluid flows through lines 28a and 28b, and/or while flowing through hoses 78a and 78b. By providing temperature readings at the thermal pads 24, temperature sensors 100a and 100b allow the thermal control unit 22 to have more accurate knowledge of the state of thermal pad 24 and its thermal characteristics.

In some embodiments of thermal control unit 22, controller 48 is configured to take one or more automatic actions in response to the detection of discrepancies between temperature readings from temperature sensors 100a and 100b that exceed one or more thresholds. Such automatic actions may be in addition to, or in lieu of, a notification to the user of the detection of such discrepancies. In one such embodiment, controller 48 is adapted to change the fluid pressure and/or fluid volume supplied to one or more of supply lines 28a in response to the detection of such a temperature discrepancy. Controller 48 may achieve this by opening, closing, or otherwise adjusting one or more of valves 44 inside of thermal control unit 22. Alternatively, some thermal pad embodiments (which will be discussed in greater detail below), include one or more valves that are controllable by thermal control unit 22 via control ports 68. When thermal control unit 22 is used with such thermal pads, control unit 22 sends a signal to the appropriate control port 68 instructing one or more valves positioned inside of the thermal pad to change their state (e.g. to open, close, or move to some position in between).

Still further, in some embodiments, thermal control unit 22 is constructed to be able to deliver fluid to zones 94 and/or thermal pads 24 that is at different temperatures. In such embodiments, controller 48 is adapted to react to detected temperature discrepancies (from sensors 100) that exceed a threshold by adjusting the temperature of the fluid supplied to one or both of the zones whose sensed temperatures are different by more than the threshold. Thus, for example, if a thermal pad 24 is being used to cool a patient and the temperature inside of first zone 94a is much cooler than the temperature inside of second zone 94b, controller 48 may lower the temperature of the fluid supplied to second zone 94b. Alternatively, controller 48 may take other actions. Such actions may vary depending upon not only the magnitude of the temperature difference between sensors 100a and 100b, but also the operational mode of thermal control unit 22, the temperature of the patient, the absolute temperature of the fluid (as measured at one or more locations), and one or more user-configured settings of thermal control unit 22.

Thermal control unit 22 is also constructed in some embodiments to allow a user to designate a priority level for one or more of the thermal pads 24. Based upon temperature and/or flow readings associated with the one or more high priority pads 24, thermal control unit 22 automatically adjusts its resources from the low priority pad(s) to the high priority pad(s) 24 if an undesired condition occurs with respect to the high priority pad(s). Such an undesired condition may include flow volume and/or temperature readings that are outside of desired ranges. The shifting of the resources includes, but is not limited to, reducing flow volumes to the low priority pad(s) via valves 44 and/or 102 (discussed below), adjusting the heat exchanger 38, pump 32, and/or other components of thermal control unit 22 based upon the readings from the high priority pad(s) instead of readings from the low priority pad(s), and/or shutting off fluid flow to the low priority pads until the desired state of the high priority pad(s) is attained. In some such embodiments, the user is able to specify how thermal control unit 22 reacts to deviations from target conditions in the one or more high priority pad(s). Still further, thermal control unit 22 is configured to allow the same priority designations discussed herein to be applied to individual zones of one or more thermal pads 24. When applied to individual zones, thermal control unit 22 is able to take the same actions discussed above when the high priority zone deviates from one or more target conditions.

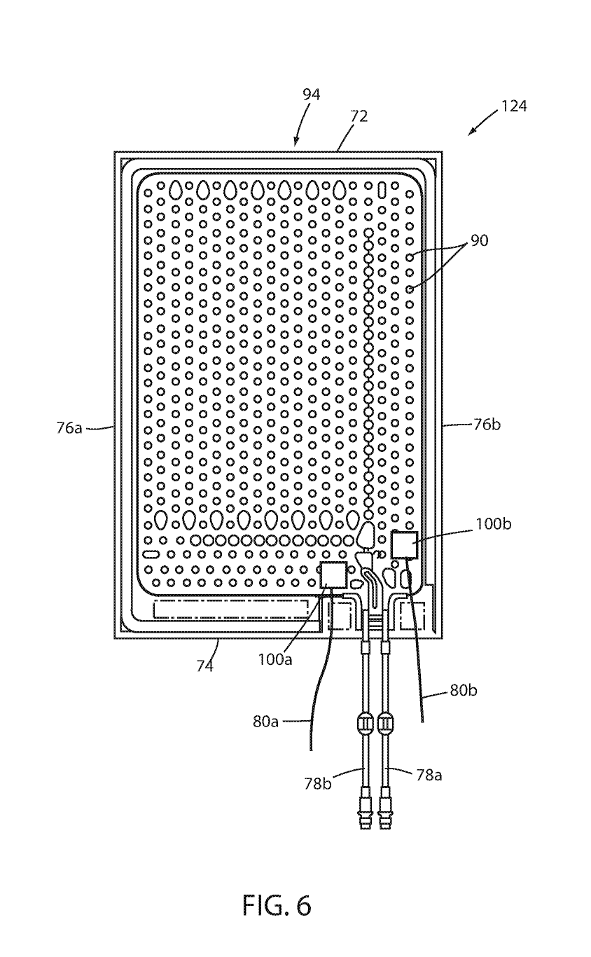

FIG. 6 illustrates an alternative embodiment of a thermal pad 124 that may be used with thermal control system 20. Thermal pad 124 includes a number of components and/or features that are the same as thermal pad 24. Those components or features that are common are labeled with the same reference numbers used to describe thermal pad 24 and, unless otherwise explicitly stated below, operate in the same manner or provide the same function as previously described. Those components or features that are different from thermal pad 24 are provided with a new reference number and described in more detail below.

Thermal pad 124 differs from thermal pad 24 in two respects. First, thermal pad 124 includes only a single zone, and thus has only one inlet hose 78a and one outlet hose 78b. Second, thermal pad 124 includes two temperature sensors 100a and 100b that are contained within the same (single) zone. In one embodiment of thermal pad 124, first temperature sensor 100a is positioned generally adjacent fluid outlet hose 78b so as to sense the temperature of the fluid as it exits, or is about to exit, thermal pad 124. Second temperature sensor 100b is positioned generally adjacent fluid inlet hose 78a so as to sense the temperature of the fluid that is entering, or has just entered, thermal pad 124. Each temperature sensor 100a and 100b is coupled to a control line 80a and 80b that are, in turn, coupled to a pair of control ports 68 on thermal control unit 22.

Thermal control unit 22 uses the temperature readings from sensors 100a and 100b of thermal pad 124 to calculate the change in fluid temperature across the thermal pad 124. That is, controller 48 calculates how much the fluid temperature changes from when the fluid first enters thermal pad 124 to when the fluid exits thermal pad 124. This provides an indication to thermal control unit 22 of how much thermal energy is being transferred to, or delivered by, thermal pad 124 (assuming a relatively steady flow rate). As noted previously, in some embodiments, thermal control unit 22 is configured to determine the flow rates for each thermal pad or zone. In such embodiments, control unit 22 uses the readings from temperature sensors 100a and 100b of thermal pad 124 to calculate how much thermal energy is being delivered to the patient, or absorbed from the patient. The calculation can either be an absolute quantity over a period of time, or it can be a rate at which the thermal energy is transferred. In some embodiments, control unit 22 displays this thermal energy transfer amount to the user.

Thermal control unit 22, in some embodiments, takes similar actions as described above with respect to thermal pad 24, if thermal control unit 22 senses that a temperature difference between sensors 100a and 100b of a first pad 124 are significantly (more than a threshold) different from the temperature difference between sensors 100a and 100b of a second pad 124. As noted above, such actions include any one or more of the following: notifying the user, adjusting a target temperature of the temperature controlled fluid, adjusting a fluid pressure and/or flow volume of the temperature controlled fluid delivered to one or both of the thermal pads 124 (such as, but not limited to, the adjustment of one or more valves), and/or other actions.

Thermal pad 124 may be used with other thermal pads 124 of similar construction, or it may be used with one or more thermal pad 24. Indeed, control system 20 is configured so that it can be used with a mixture of one or more thermal pads 24 and/or 124 (and/or one of the other thermal pad embodiments discussed below) and/or with other thermal pads. Regardless of the homogeneity or heterogeneity of the types of thermal pads coupled to thermal control unit 22, thermal control unit 22 is adapted, in some embodiments, to individually control one or more of the pads, or one or more of the zones of the pads. That is, thermal control unit 22 controls any one or more of the following characteristics of a thermal control pad or zone in a manner that may be different from the other pads or zones coupled to thermal control unit 22: the temperature of the fluid delivered to the pad or zone by thermal control unit 22, the flow rate of the fluid delivered to the pad or zone, and/or one or more valves associate with the pad or zone. Thermal control unit 22 therefore gives the users the ability to individually, and uniquely (if desired), control the thermal therapy that is delivered to the patient at each zone and/or at each thermal pad.

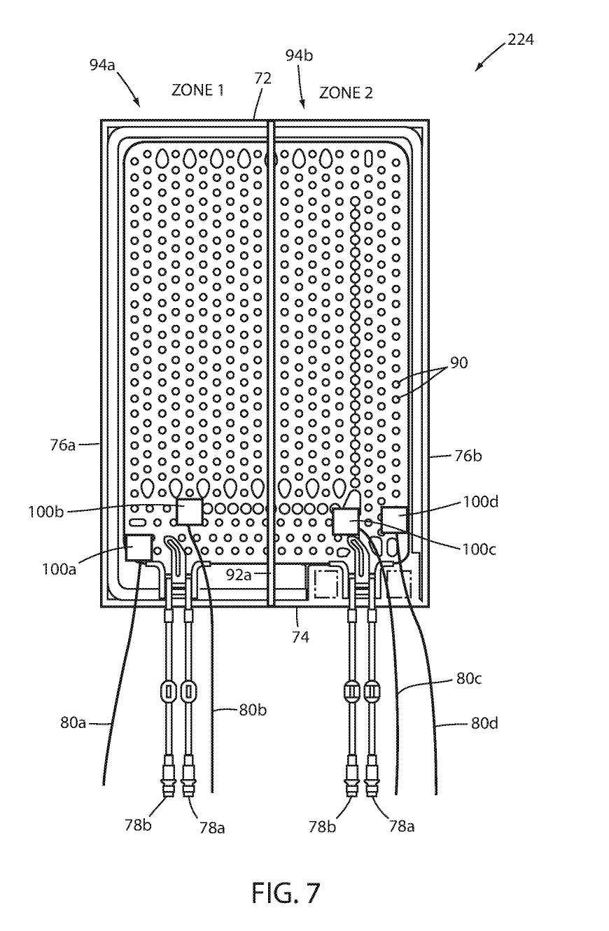

FIG. 7 illustrates an alternative embodiment of a thermal pad 224 that may be used with thermal control system 20. Thermal pad 224 includes a number of components and/or features that are the same as thermal pad 24 and/or 124. Those components or features that are common are labeled with the same reference numbers used to describe thermal pads 24 and/or 124, and unless otherwise explicitly stated below, operate in the same manner or provide the same function as previously described. Those components or features that are different from thermal pad 24 and/or 124 are provided with a new reference number and described in more detail below.

Thermal pad 224 is similar to thermal pad 124 but includes multiple zones 94a and 94b. Each zone 94 includes a pair of temperature sensors 100. Specifically, zone 94a includes temperature sensors 100a and 100b, and zone 94b includes temperature sensors 100c and 100d. Temperature sensors 100a and 100b operate in the same manner, and may be constructed in the same manner, as temperature sensors 100a and 100b of thermal pad 124, except that temperature sensors 100a and 100b of thermal pad 224 measure fluid temperatures for a particular zone (94a) of thermal pad 224 (rather than for the entire pad, as with thermal pad 124). Further, thermal control unit 22 uses the outputs from temperature sensors 100a and 100b of thermal pad 224 in any of the same manners discussed above that it uses the outputs from temperature sensors 100a and 100b of thermal pad 124.

Temperature sensors 100c and 100d operate in the same manner, and may be constructed in the same manner, as temperature sensors 100a and 100b of thermal pad 124, except that temperature sensors 100c and 100d of thermal pad 224 measure fluid temperatures for a particular zone (94b) of thermal pad 224 (rather than for the entire pad, as with temperature sensors 100a and 100b of thermal pad 124). Each temperature sensor 100a, b, c, and d of thermal pad 224 is coupled to a corresponding control line 80a, b, c, and d, respectively. Controls lines 80a-d, in turn, are coupled to four of the control ports 68 of thermal control unit 22. Thermal pad 224 may be used in a thermal control system 20 that also utilizes one or more different types of thermal pads when controlling the temperature of a patient (e.g. thermal pads 24, 124, and/or others), or it may be used in a thermal control system 20 that utilizes only similar thermal pads 224. Of course, in some embodiments, thermal control system 20 only includes a single thermal pad.

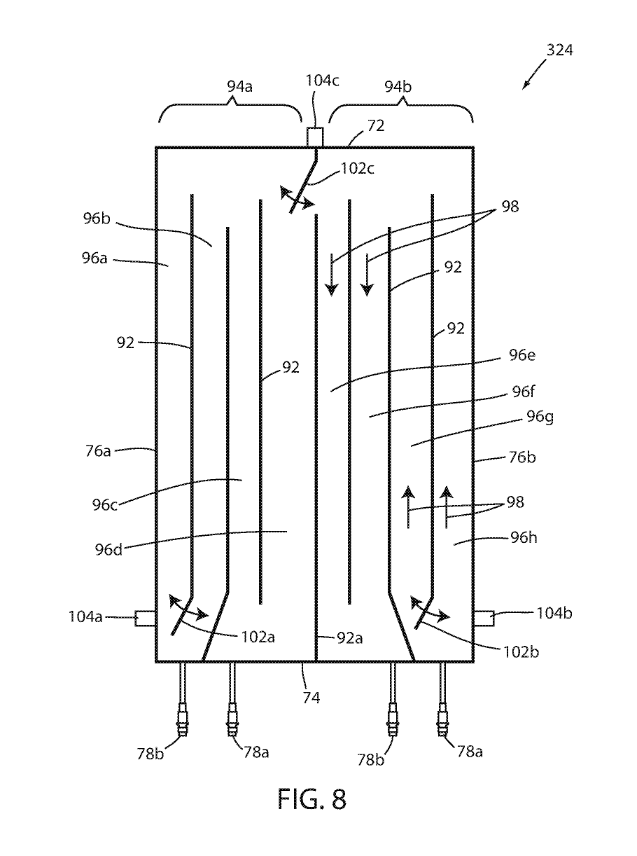

FIG. 8 illustrates another alternative embodiment of a thermal pad 324 that may be used with thermal control system 20. Thermal pad 324 includes a number of components and/or features that are the same as thermal pads 24, 124, and/or 224. Those components or features that are common are labeled with the same reference numbers used to describe thermal pads 24, 124, and/or 224, and unless otherwise explicitly stated below, operate in the same manner or provide the same function as previously described. Those components or features that are different from thermal pads 24, 124, and/or 224 are provided with a new reference number and described in more detail below.

As seen in FIG. 8, thermal pad 324 includes two zones 94a and 94b. Each zone 94 includes a plurality of internal walls 92 that define a plurality of fluid channels 96a-h. First zone 94a includes fluid channels 96a-d and second zone 94b includes fluid channel 96e-h. Fluid flows in channels 96a-h in the directions indicated by arrows 98. It will be understood by those skilled in the art that different shapes of fluid channels 96a-h can be used. Further, it will be understood by those skilled in the art that in some embodiments, one or more of fluid channels 96a-h may have a shape that is different from one or more of the other fluid channels 96a-h.

In addition to multiple fluid channels 96, thermal pad 324 also includes a plurality of valves 102. Valves 102 control the flow of fluid through one or more of the fluid channels 96, as will be described in more detail below. Each valve 102 includes an associated control port 104. Control ports 104 are coupled to wires, cables, or other similar structures that are in communication with thermal control unit 22 via control ports 68. In some embodiments, control ports 104 may be wireless transceivers that wirelessly communicate with wireless transceivers on thermal control unit 22. Regardless of the media used to communicate between thermal pad 324 and thermal control unit 22, thermal control unit 22 sends control signals to thermal pad 324 that control the position of valves 102.

As shown in FIG. 8, a first valve 102a is positioned in first zone 94a generally adjacent outlet hose 78b. First valve 102a controls the relative amount of fluid that exits from pad 324 via channels 96a and 96b. First valve 102a is movable between a first blocking position that completely stops fluid flow through first channel 96a and a second blocking position that completely stops fluid flow through second channel 96b, as well as a plurality of intermediate positions that allow varying amounts of fluid to flow through first and second channels 96a and 96b. First valve 102a is controlled by signals received at first control port 104a.

A second valve 102b is positioned in second zone 94b generally adjacent inlet hose 78a. Second valve 102b controls the relative amount of fluid that enters zone 94b of pad 324 via channels 96g and 96h. Second valve 102b is movable between a first blocking position that completely stops fluid from entering fluid channel 96g and a second blocking position that completely stops fluid from entering fluid channel 96h, as well as a plurality of intermediate positions that allow varying amounts of fluid to flow into channels 96g and 96h. Second valve 102b is controlled by signals received at second control port 104b.

Thermal pad 324 also includes a third valve 102c that is an inter-zone valve. That is, third valve 102 controls the amount of fluid that is able to pass between first zone 94a and second zone 94b. Third valve 102c is movable between a blocking position that maintains fluid isolation between zones 94a and 94b and an open position in which valve 102c is completely open to allow fluid to freely flow therethrough between zones 94a and 94b. Third valve 102c is also movable to a plurality of intermediate positions that partially restrict fluid flow between zones 94a and 94b. Third valve 102c is controlled by signals received at third control port 104c.

It will be understood by those skilled in the art that the number of valves 102 and their position within thermal pad 324 may be varied widely from that illustrated in FIG. 8. For example, in some modified embodiments, thermal pad 324 includes valves 102a and 102b that are both positioned adjacent inlet hoses 78a of each zone, or that are both positioned near outlet hoses 78b of each zone, rather than in the locations shown in FIG. 8 wherein valve 102a is positioned adjacent outlet hose 78b of one zone and valve 102b is positioned adjacent inlet hose 78a of the other zone. In other modified embodiments, thermal pad 324 eliminates inter-zone valve 102c, or includes more than one inter-zone valve 102c. In yet another alternative, thermal pad 324 includes fewer valves 102 than the three shown in FIG. 8. Thermal pad 324 may also be modified to include only a single zone or more than the two zones shown in FIG. 8.

Still further, thermal pad 324 may be modified to include one or more valves 102 that, instead of controlling a relative amount of fluid flowing through two or more channels, such as is illustrated in FIG. 8, are configured to control the amount of fluid flowing through a single channel. In such a modified embodiment, the valve 102b shown in FIG. 8, for example, could be replaced by a pair of valves 102, one of which controls how much fluid flows through channel 96g independent of the other one which controls how much fluid flows through channel 96h. As yet another alternative, thermal pad 324 may be constructed such that one or more of the zones 94a, b has more channels 94 and/or valves 102 than what are illustrated in FIG. 8, or has fewer channels 96 and/or valves 102 than what are illustrated in FIG. 8.

Additionally, although not illustrated in FIG. 8, thermal pad 324 may include one or more temperature sensors 100. In some embodiments, thermal pad 324 includes a single temperature sensor 100 for each zone 94a and 94b, similar to thermal pad 24. In other embodiments, thermal pad 324 includes both an inlet temperature sensor 100 and an outlet temperature sensor 100 for each zone, similar to thermal pad 224. In still other embodiments, still other numbers and/or locations of temperature sensors 100 may be implemented.

Regardless of the specific number and position of valves 102 and zones 94, thermal control unit 22 controls the valves based upon temperature readings from the temperature sensors 100, the flow rates to each zone (if thermal control unit 22 includes flow meters for individual zones), the temperature difference between one or more of the temperature sensors 100 and/or temperature sensors 42 and/or 66, the target fluid temperature, the target patient temperature, and/or one or more user-configurable settings of thermal control unit 22.

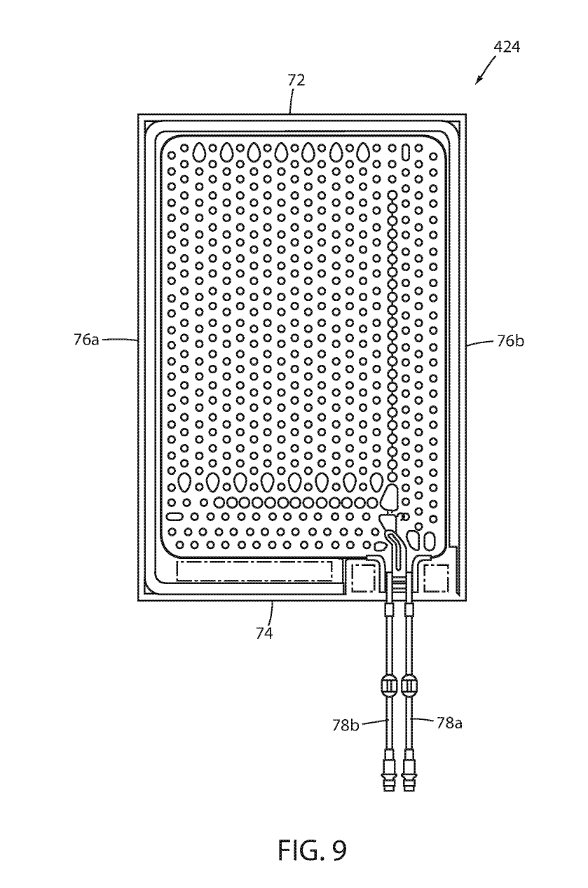

FIGS. 9-11 illustrate another alternative embodiment of a thermal pad 424 that may be used with thermal control system 20. Thermal pad 424 includes a number of components and/or features that are the same as thermal pads 24, 124, 224, and/or 324. Those components or features that are common are labeled with the same reference numbers used to describe thermal pads 24, 124, 224, and/or 324, and unless otherwise explicitly stated below, operate in the same manner or provide the same function as previously described. Those components or features that are different from thermal pads 24, 124, 224, and/or 324 are provided with a new reference number and described in more detail below.

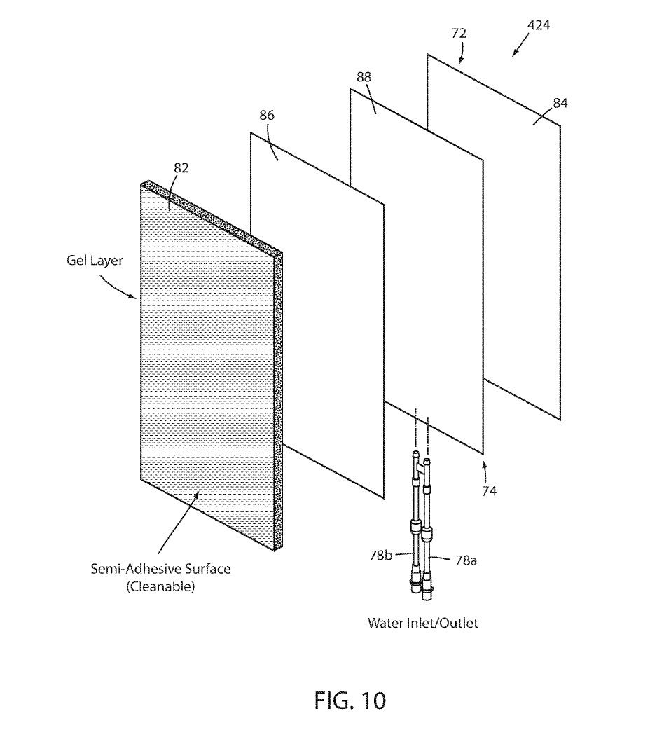

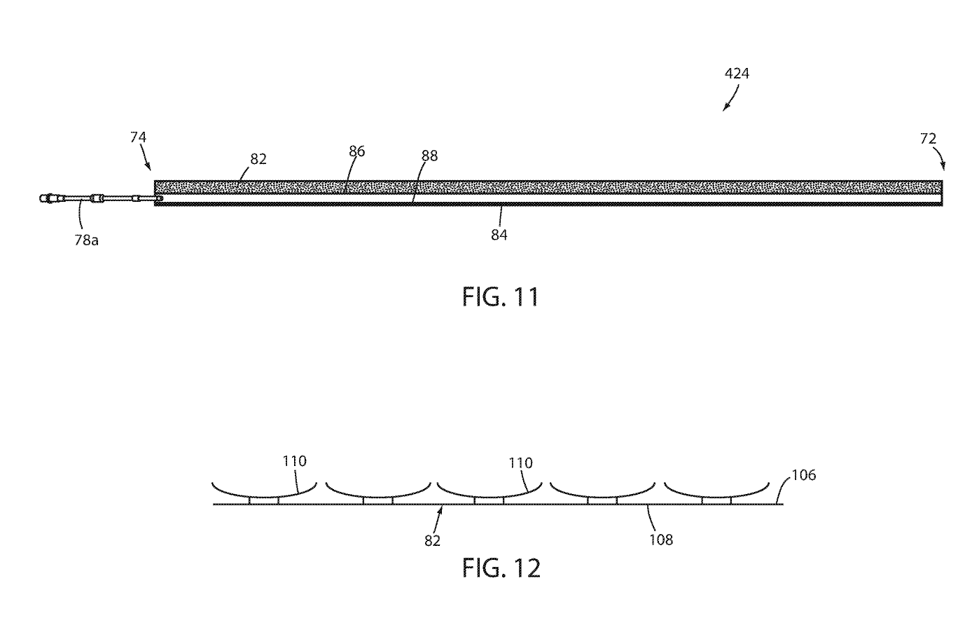

As shown more clearly in FIGS. 10 and 11, thermal pad 424 includes an interior layer 82, an exterior layer 84, and first and second intermediate layers 86 and 88. As with thermal pad 24, first intermediate layer 86 and second intermediate layer 88 are bonded at their perimeter (and potentially one or more interior locations 90). The space between intermediate layers 86 and 88 defines a chamber for the temperature controlled fluid to flow. The chamber may include one or more walls 92 defining one or more fluid channels 96. Further, although not illustrated in FIGS. 9-11, thermal pad 424 may include one or more valves 102 and/or one or more temperature sensors 100 incorporated therein in any of the manners previously described.

Interior layer 82 of thermal pad 424 is made of a gel material having higher thermal conductivity than air. The gel material is adapted to releasably adhere to the skin of a patient in order to provide and maintain close physical contact of thermal pad 424 with the patient. The particular gel material used for interior layer 82 may vary. In some embodiments, the gel is a urethane gel. The specific chemical composition of the urethane gel can be adjusted to change the adhesive properties of the side of interior layer 82 that contacts the patient's skin. Gel layer 82 may be secured to first intermediate layer 86 by RF welding, lamination, by being poured thereon, or by other means. Regardless of the specific gel used and the specific manner it is secured to first intermediate layer 86, the gel should provide suitable adhesion to the surface of the patient's skin in order to resist physical separation between the pad 424 and the patient, yet not be so resistant to physical separation so as to cause discomfort to the patient when the pad 424 is subsequently removed. Gel layer 82 of thermal pad 424 includes an exterior surface that contacts the patient and that, in some embodiments, is cleanable with water and/or mild surfactants, thereby allowing the thermal pad 424 to be re-used, if desired. In some embodiments, one or more antibacterial materials are incorporated into the gel in order to resist the growth of bacteria on the patient's skin where the pad 424 is placed. Alternatively, or additionally, the gel layer may include a skin conditioner (e.g. lanolin, aloe, etc.) that helps prevent chapping, chafing, or other types of skin degradation.

FIG. 12 illustrates an alternative interior layer 82 that may be used on thermal pad 424 in place of gel layer 82 and/or that may be used on any of thermal pads disclosed herein in place of the other interior layers 82 described herein. Interior layer 82 of FIG. 12 includes an interior surface 106 and an exterior surface 108. Interior surface 106 faces toward the patient while exterior surface 108 faces away from the patient. Exterior surface 108 is bonded to a first intermediate layer 86 (not shown in FIG. 12). Interior surface 106 includes a plurality of craters 110 defined on it that act as suctions cups when contacting the skin of the patients. Craters 110 therefore releasably adhere to the patient and help maintain physical contact between the patient's skin and the thermal pad (of which interior layer 82 is a part).

The shape, size, and number of craters 110 may vary. In general, craters 110 act in a similar manner to the craters found on many conventional shower or bath mats that releasably stick the mat to the floor of the shower or bath. That is, craters 110 are defined by resiliently flexibly material that, when engaged with the person's skin, are designed to maintain a negative gauge pressure therein. The negative gauge pressure urges the patient's skin and craters together. Craters 110 are made from any material that has suitable characteristics for creating negative gauge pressure or suction between the layer 82 and the patient's skin and that has good thermal transfer properties for transferring thermal energy between the patient's skin and the thermal pad.

It will be understood by those skilled in the art that many variations may be made to the thermal pads described herein beyond the specific modifications described above. For example, and not by way of limitation, the temperatures sensors 100 have been primarily described herein as being inside of thermal pads 24 where they may come into direct contact with the circulating fluid, but it will be understood that they may be placed elsewhere, such as on an exterior surface of the thermal pads. In some of these modified embodiments, the temperature sensors 100 are used to measure the patient's temperature. Still further, some thermal pads may include both internal and external temperature sensors wherein the internal temperature sensors measure the temperature of the circulating fluid and the external temperature sensors measure the temperature of the patient and/or the ambient surroundings of the thermal pad. In other embodiments, any one or more of the valves 102 used in the thermal pads may be replaced with pressure operated valves that are not controlled by signals received through a corresponding control port 104. In such embodiments, the thermal pads may omit control ports 104 for the pressure operated valves.

It will also be understood by those skilled in the art that although the thermal pads described herein have been described as being used in conjunction with thermal control unit 22, and vice versa, that the thermal control unit 22 can be used with other types of thermal pads than those described herein and that the thermal pads can be used with other types of thermal control units than the thermal control unit 22 described herein. As one example, if thermal pads 24 include one or more temperature sensors 100, they may be used with, in some embodiments, a thermal control unit that does not include one or more of the temperature sensors 42 and/or 66. In such embodiments, the controller uses the temperature readings from the thermal pads to control the heat exchanger 38 and/or pump 32. Still other modifications are possible.

Still further, it will be understood that although the flow of fluid to the thermal pads described herein has been described herein as being controlled by valves 44 positioned in thermal control unit 22 and/or valves 102 positioned inside of the thermal pads, the flow of fluid can also be controlled by one or more valves positioned between thermal control unit 22 and the attached thermal pads, such as by one or more valves coupled to hoses 26, or to one or more structures positioned intermediate control unit 22 and the thermal pads.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A thermal pad for controlling a patient's temperature, the thermal pad comprising:
a body adapted to be placed in contact with the patient, the body defining an interior in which fluid circulates, the body comprising a fluid inlet and a fluid outlet, the fluid inlet adapted to receive the fluid from a thermal control unit adapted to control a temperature of the fluid, and the fluid outlet adapted to return the fluid to the thermal control unit;
a first temperature sensor coupled to the body and adapted to detect a first temperature at a first location;
a second temperature sensor coupled to the body and adapted to detect a second temperature at a second location; and
a temperature output for reporting the temperature of the first and second temperature sensors to the thermal control unit.

2. The thermal pad of claim 1 wherein the body includes a first channel coupling the fluid inlet to the fluid outlet.

3. The thermal pad of claim 2 further comprising:
a second fluid inlet;
a second fluid outlet; and
a second channel coupling the second fluid inlet to the second fluid outlet, the second channel separated from the first channel such that fluid in the first channel does not mix with fluid in the second channel while inside the thermal pad.

4. The thermal pad of claim 3 further comprising:
a third temperature sensor positioned adjacent the second fluid outlet and adapted to detect a temperature of the fluid exiting the thermal pad from the second fluid outlet;
a fourth temperature sensor positioned adjacent the second fluid inlet and adapted to detect a temperature of the fluid entering the thermal pad from the second fluid inlet; and
wherein the temperature output also reports the temperature of the third and fourth temperature sensors to the thermal control unit.

5. The thermal pad of claim 1 wherein the body includes an interior surface adapted to contact the patient and an exterior surface adapted to face away from the patient, the interior surface including a plurality of craters adapted to create suction when the interior surface is applied to a skin of the patient, the suction releasably retaining the interior surface against the patient's skin.

6. The thermal pad of claim 5 wherein the interior surface includes no adhesive.

7. The thermal pad of claim 1 wherein the body includes an interior surface adapted to contact the patient and an exterior surface adapted to face away from the patient, the interior surface including a gel layer adapted to directly contact a skin of the patient.

8. The thermal pad of claim 1 further comprising:
a plurality of channels defined in the body, each of the channels in fluid communication with the fluid inlet to the fluid outlet; and
a valve adapted to control an amount of fluid flowing through at least one of the plurality of channels.

9. The thermal pad of claim 1 further comprising a second fluid inlet and a valve adapted to control an amount of fluid flowing into at least one of the fluid inlet and the second fluid inlet.

10. The thermal pad of claim 1 wherein the first temperature sensor is positioned adjacent the fluid outlet and adapted to detect a temperature of the fluid exiting the thermal pad, and the second temperature sensor is positioned adjacent the fluid inlet and adapted to detect a temperature of the fluid entering the thermal pad.

11. A thermal control system for controlling a patient's temperature, the thermal control system comprising:
a thermal pad according to claim 1; and
a thermal control unit, the thermal control unit comprising:
a first fluid outlet adapted to fluidly couple to a first fluid supply line, the first fluid supply line adapted to couple to the fluid inlet of the thermal pad;
a first fluid inlet adapted to fluidly couple to a first fluid return line, the first fluid return line adapted to couple to the fluid outlet of the thermal pad;
a heat exchanger;
a pump for circulating fluid from the first fluid inlet through the heat exchanger and to the first fluid outlet; and
a controller adapted to receive a first temperature reading and a second temperature reading from the temperature output of the thermal pad, the first temperature reading coming from the first temperature sensor coupled to the body of the thermal pad and the second temperature reading coming from the second temperature sensor coupled to the body of the thermal pad, the controller further adapted to control the heat exchanger based upon the first and second temperature readings.

12. A thermal pad for controlling a patient's temperature, the thermal pad comprising:
a body adapted to be placed in contact with the patient, the body defining an interior in which fluid circulates, the body comprising a fluid inlet and a fluid outlet, the fluid inlet adapted to receive the fluid from a thermal control unit adapted to control a temperature of the fluid, and the fluid outlet adapted to return the fluid to the thermal control unit;
a channel defined in the body and in fluid communication with the fluid inlet to the fluid outlet; and
a valve adapted to control an amount of fluid flowing through the channel.

13. The thermal pad of claim 12 further including a port adapted to receive a control signal for controlling the valve.

14. The thermal pad of claim 12 wherein the body comprises a plurality of channels, and a first one of the plurality of channels is associated with a first zone of the thermal pad and a second one of the plurality of channels is associated with a second zone of the thermal pad, and the valve controls what proportion of the fluid from the fluid inlet is directed to the first zone versus the second zone.

15. The thermal pad of claim 12 wherein the valve is positioned at the fluid inlet and controls the amount of fluid flowing through the channel.

16. The thermal pad of claim 15 wherein the valve is a pressure operated valve controlled by a pressure of the fluid.

17. The thermal pad of claim 16 further comprising a second fluid inlet and a second valve positioned at the second fluid inlet.

18. The thermal pad of claim 17 wherein the second fluid inlet is fluidly coupled to a second channel and a second fluid outlet.

19. The thermal pad of claim 18 wherein the second valve is a pressure operated valve controlled by a pressure of the fluid.

20. The thermal pad of claim 12 wherein the body includes an interior surface adapted to contact the patient and an exterior surface adapted to face away from the patient, the interior surface including a gel layer adapted to directly contact a skin of the patient.

21. The thermal pad of claim 12 wherein the body includes an interior surface adapted to contact the patient and an exterior surface adapted to face away from the patient, the interior surface including a plurality of craters adapted to create suction when the interior surface is applied to a skin of the patient, the suction releasably retaining the interior surface to the patient's skin.

22. A thermal control system for controlling a patient's temperature, the thermal control system comprising:
a thermal pad according to claim 12; and
a thermal control unit, the thermal control unit comprising:
a first fluid outlet adapted to fluidly couple to a first fluid supply line, the first fluid supply line adapted to couple to the fluid inlet of the thermal pad;
a first fluid inlet adapted to fluidly couple to a first fluid return line, the first fluid return line adapted to couple to the fluid outlet of the thermal pad;
a heat exchanger;
a pump for circulating fluid from the first fluid inlet through the heat exchanger and to the first fluid outlet;
a temperature sensor adapted to detect a temperature of fluid returning from the first fluid inlet;
a first valve for controlling an amount of fluid flowing to the first fluid outlet; and
a controller adapted to control the first valve based at least in part upon the temperature sensed by the temperature sensor.

23. The thermal control unit of claim 22 wherein the controller is further adapted to receive a temperature reading from a second temperature sensor integrated into the thermal pad, wherein the controller is further adapted to control the valve of the thermal pad based at least in part upon the temperature reading received from the second temperature sensor.

24. The thermal control unit of claim 22 wherein the controller adjust an amount of pressure in the fluid supplied to the first fluid outlet based at least in part upon the temperature sensed by the temperature sensor.
